(19) United States
(12) Patent Application Publication
(10) Pub. No.: US 2007/0281961 A1
(43) Pub. Date: Dec. 6, 2007

(54) Modified Compositions And Methods For Enhancing Brain Function

(75) Inventor: Josh Reynolds, Laguna Beach, CA (US)

Correspondence Address: FISH & ASSOCIATES, PC;ROBERT D. FISH, 2603 Main Street, Suite 1050, Irvine, CA, 92614-6232, US

(73) Assignee: BRITE AGE, Laguna Hills, CA (US)

(21) Appl. No.: 11/758,173

(22) Filed: Jun. 5, 2007

Related U.S. Application Data
(60) Provisional application No. 60/803,943, filed on Jun. 5, 2006.
(60) Provisional application No. 60/820,201, filed on Jul. 24, 2006.

(51) Int. Cl. A61K 31/473; A61K 31/4188; A61K 31/385; A61K 31/205

(52) U.S. Cl. 514/290; 514/440; 514/554; 514/393

(57) ABSTRACT

Compositions and methods are provided that enhance cognition in a human to which the composition is orally administered. Clinical studies have proven that contemplated compositions achieve specific improvements in cognition using a modified nootropic formulation having six active ingredients at or near threshold active dosages. Most preferably, the active ingredients are huperzine A, vinpocetine, acetyl-L-carnitine, alpha lipoic acid, rhodiola, and biotin at typically less than 2300 mg per daily dosage.

This application claims priority to our copending U.S. provisional applications with the Ser. Nos. 60/803,943, filed Jun. 5, 2006, and 60/820,201, filed Jul. 24, 2006.

### FIELD OF THE INVENTION

The field of the invention is nutritional supplements and methods therefore, especially as they relate to enhancers of cognition and mood.

### BACKGROUND OF THE INVENTION

Numerous approaches are known in the art to enhance mood and cognitive performance in normal individuals, including pharmaceutical interventions, aerobic exercise and certain cognitive training programs. More recently, certain nutraceutical agents, such as, ginkgo biloba, and multi-agent compounds have claimed cognitive enhancing effects. Unfortunately, most of those agents and compounds make claims based on mere inclusion of one or more individual ingredients whose clinically demonstrated efficacy level(s), or minimal therapeutic threshold amount(s), are typically not achieved in the proposed multi-agent compound.

Moreover, the few isolated compounds claiming one or more cognitive effects that have been subjected to well controlled (e.g., randomized, double blind, placebo controlled) clinical trials in relatively significant sample sizes (e.g., >50) have only shown clinical effect in selected populations (e.g., an older population, cognitively impaired, abnormal, or low normal sub-population), and may therefore have no significant effect in a healthy population of relatively wide age range. In general, there have been few, if any, qualified studies showing single or multi-agent compounds positively and significantly effecting both mood and cognitive status in a healthy, broad age range population.

In other instances, various supplements and formulations comprising a multiplicity of allegedly active ingredients are marketed as nootropics, or cognitive enhancing agents. For example, the commercially available “Focus Factor” formulation sports over 30 ingredients, while the commercially available “Brain Lightning” formulation has nearly 20 ingredients. Most typically, such formulations are marketed as including multiple active ingredients with respective specific effects, and therefore often suggest that multiple active ingredients will provide additive, or even synergistic beneficial effects.

Unfortunately, there is no clinical trial for such formulations that affirms such suggested results. Indeed, the beneficial effects of selected ingredients, which individually show cognitive benefits, may possibly even be canceled out when combined by sensory or metabolic over-stimulation. Various examples for ineffective multi-ingredient formulations are provided in my copending U.S. patent application with the title “Compositions And Methods For Enhancing Brain Function” (inventor J. Reynolds), filed on Jun. 5, 2007. There (and further below herein), it is clearly demonstrated that addition of certain ingredients to a clinically proven effective formulation typically abrogates the activity of such formulation, which is contrary to what is typically promoted in advertising of other known multi-ingredient formulations.

Therefore, while numerous compositions and methods for cognitive enhancement are known in the art, all or almost all of them suffer from one or more disadvantages. Thus, there is still a need to provide improved nootropic compositions and methods, for improvements in mood and cognitive function in both abnormal, low to normal, and normal to high functioning general population groups.

### SUMMARY OF THE INVENTION

The present invention is directed to compositions and methods for nootropic nutritional supplements to enhance cognitive function and mood. Surprisingly, clinical tests have revealed that the compositions contemplated herein have statistically significant distinct cognitive and mood enhancing effects when compared to observed effects of a seemingly similar composition. Even more unexpectedly, the addition of multiple selected ingredients to a formulation having proven clinical effect at respective contemplated relative amounts did not abrogate the clinical efficacy, but shifted the efficacy profile in a distinct manner.

In one aspect of the inventive subject matter, contemplated nutritional supplements for enhancing cognitive function preferably include a group of six active ingredients: Huperzine A, vinpocetine, acetyl-L-carnitine, alpha lipoic acid, rhodiola, and biotin. It is also preferred that the huperzine A, the vinpocetine, and the acetyl-L-carnitine are present in a ratio of x:y:z, wherein x is between 0.8 and 1.2, y is between 80 and 120, and z is between 8,000 and 12,000, and/or that the ratio between the acetyl-L-carnitine and the alpha lipoic acid, the rhodiola, and the biotin is between 2:1 and 20:1, between 2:1 and 20:1, and between 20,000:1 and 200:1, respectively.

Viewed from a different perspective, contemplated supplements comprise in a daily dose huperzine A in an amount between 100-200 mcg, vinpocetine in an amount between 10-30 mg, and acetyl-L-carnitine in an amount between 1,500-2,000 mg, and/or in a daily dose alpha lipoic acid in an amount between 250-500 mg, rhodiola in an amount between 200-400 mg, and biotin in an amount between 250-750 mcg. In addition to the active ingredients, inactive ingredients may be added, including carriers, binders, excipients, and/or dyes. Therefore, the huperzine A, vinpocetine, and acetyl-L-carnitine together account for at least 50 wt % of a dosage unit of the supplement (excluding inactive ingredients) in contemplated formulations. Alternatively or additionally, the supplement may be formulated for oral administration such that the group of active ingredients accounts for at least 85 wt % (and more preferably at least 90 wt %) of a dosage unit of the supplement (excluding inactive ingredients). While not limiting to the inventive subject matter, it is generally preferred that the daily dosage of the supplement is equal or less than 2,500 mg, and more preferably equal or less than 2,300 mg. Furthermore, where desired, an information may be associated with the nutritional supplement that states that the nutritional supplement enhances at least one of working memory, general intelligence, attention, and mood.

Therefore, in a further aspect of the inventive subject matter, a method of assisting enhancement of cognitive function in a person using a nutritional supplement will include a step of providing a composition that includes a group of active ingredients consisting of huperzine A, vinpocetine, acetyl-L-carnitine, alpha lipoic acid, rhodiola, and biotin, wherein the supplement is formulated for oral administration such that the active ingredients together account for at least 90 wt % of a dosage unit of the supplement, excluding inactive ingredients. In yet another step, information is provided that the supplement in a clinical trial was effective to improve cognitive function (and especially working memory, general intelligence, attention, and/or mood). In still further contemplated methods, an interactive tool that allows validation of the efficacy of the supplement and/or proper personal dosing of the supplement can be provided. With respect to preferred nutritional supplements, the same considerations as provided above apply.

Various objects, features, aspects and advantages of the present invention will become more apparent from the following detailed description of preferred embodiments of the invention.

### DETAILED DESCRIPTION

The inventor has discovered that specific additional nutritionally safe compounds can be combined with a clinically proven nootropic composition to alter specific cognitive enhancement effects without abrogating statistically significant improvements in cognition and mood of such nootropic compositions. Such finding is particularly unexpected as numerous alternative and seemingly similar formulations have no statistically significant and clinical effect on cognition and mood (see below).

It should further be especially recognized that contemplated compositions are not only effective for improvement of cognitive states and status in a healthy population within a wide age range (clinically proven effective for ages 25-65), but also for a patient pool ranging from declining more rapidly than normal, or accelerated stages of decline, to pre-dementia states (e.g., MCI−mild cognitive impairment), and even MCI precursor states (e.g., AAMI).

More particularly, contemplated compositions have been demonstrated to improve with high statistical significance several parameters on the Raven Progressive Matrices (especially working memory and general intelligence), attention (e.g., Digit Vigilance Error reduction), and mood (particularly reduction in stress). The formulations according to the inventive subject matter have also produced results strongly suggesting (i.e., approaching statistical significance) improvements in working memory (e.g., reduction in Spatial Working Memory Errors and increase in Numerical Working Memory Accuracy), in memory consolidation (e.g., Word Recognition Accuracy Original Stimuli), and mood improvements (particularly tension). Such activity profile is entirely unexpected as the activity profile of the same formulation lacking alpha lipoic acid, Rhodiola, and biotin is significantly different (see below).

It should be noted that heretofore known nootropic formulations included a large range of ingredients (e.g., U.S. Pat. No. 6,964,969 listing 47 ingredients) with entirely uncharacterized interactions. Clinical information on cognitive enhancement of such formulations was only available for isolated ingredients. Therefore, and as pointed out in more detail further below, the presumed effect of such formulations was based on a summarization of known effects of individual ingredients, which is in most if not all cases inconsistent with the actual effect. On the other hand, certain supplements have been tested and were found effective in a specific manner (e.g., Eur. J. Pharmacol. 2000; 398(1):56-72 where improvement of cognitive function in rats with chronic cerebral hypoperfusion is reported). As a consequence, these supplements are often marketed with unsubstantiated and/or overextended claims with regard to their alleged effect on human cognition as the reported and specific effects often fail to translate into specific and measured advantages in human.

In one exemplary and preferred formulation, a nutritional supplement or pharmaceutical composition is prepared that includes a therapeutically effective daily dosage of (1) acetyl-L-carnitine (preferably 1000-3000 mg/d, more preferably 1500-2000 mg/d, and most preferably about 1500 mg/d), (2) Vinpocetine (preferably 5-45 mg/d, more preferably 10-30 mg/d, and most preferably about 15 mg/d), (3) Huperzine A (preferably 50-300 mcg/d, more preferably 100-200 mcg/d, and most preferably about 150 mcg/d), (4) alpha lipoic acid (preferably 100-600 mg/d, more preferably 250-500 mg/d, and most preferably about 400 mg/d), (5) Rhodiola rosea powdered root extract [standardized to 3 wt % rosavin] (preferably 100-600 mg/d, more preferably 200-400 mg/d, and most preferably about 300 mg/d), and (6) biotin (preferably 100-5000 mcg/d, more preferably 250-750 mcg/d, and most preferably 500 mcg/d).

Additional ingredients for contemplated formulations are also contemplated, however, not preferred if such ingredients are active ingredients (i.e., those having actual or suspected nootropic effect, or are otherwise physiologically relevant). For example, additional active ingredients include minerals, vitamins, anti-oxidants, MDAE, etc.). In most instances where additional active ingredients are added, such active ingredients are present in relatively small quantities. For example, additional active ingredients may be present in amounts of between 0.001-20 wt % of a dosage unit (excluding inactive ingredients), more typically between 0.01-10 wt % of a dosage unit (excluding inactive ingredients), and most typically between 0.001-1 wt % of a dosage unit (excluding inactive ingredients).

It should be further appreciated that, depending on the actual formulation, contemplated compositions will also include various inactive ingredients. The term “inactive ingredients” as used herein refers to those ingredients that are added to improve or stabilize formulation and/or that have no substantial physiological relevance or nootropic effect. Therefore, and among other choices, contemplated inactive ingredients include fillers, binders, disintegrants, lubricants, and dyes.

With respect to the formulation, it is generally preferred that the compositions according to the inventive subject matter are formulated for oral administration in solid or liquid form, including powders suitable for making a drink. For example, solid forms preferably include tablets, pills, dragees, capsules, and softgels, all of which may have an enteric coating to avoid or reduce gastric upset. Further solid formulations include powdered formulations, which may include flavors and other ingredients to form a dissolvable/dispersible powder that can be mixed into a drink or water. Alternatively, contemplated formulations may also be in liquid format for administration as a drink, or as drops from an eye dropper or other liquid dispenser.

In particularly preferred aspects, the supplement according to the inventive subject matter includes huperzine A, vinpocetine, and acetyl-L-carnitine, wherein the huperzine A, the vinpocetine, and the acetyl-L-carnitine are present in a ratio of x:y:z. X is preferably between 0.8 and 1.2 (and even more preferably between 0.9 and 1.1), y is between 80 and 120 (and even more preferably between 90 and 110), and z is between 8,000 and 12,000 (and even more preferably between 9,000 and 11,000). Additionally, or alternatively, it is preferred that the ratio between acetyl-L-carnitine and the alpha lipoic acid, the rhodiola, and the biotin is between 2:1 and 20:1, between 2:1 and 20:1, and between 20,000:1 and 200:1, respectively. Therefore, in most formulations, huperzine A, vinpocetine, and acetyl-L-carnitine will together account for at least 50 wt % (more typically least 60 wt %, and most typically least 65 wt %) of a dosage unit of the supplement, excluding inactive ingredients. Together with the additional active ingredients (alpha lipoic acid, Rhodiola, and biotin), it is therefore preferred that contemplated supplements are formulated for oral administration such that the six active ingredients account for at least 85 wt %, more preferably at least 90 wt %, and most preferably at least 95 wt % (all excluding inactive ingredients) of a dosage unit of the supplement. In most cases, it is preferred that the daily dosage of the supplement is equal or less than 4,000 mg, more typically equal or less than 2,500 mg, and most typically equal or less than 2,300 mg. Of course, it should be recognized that the daily dosage of the supplement can be divided into multiple sub-dosages that may be administered at the same time or distributed over a day. For example, a daily dosage of about 2,400 mg may be administered as two tablets of 400 mg each at three different times of the day.

Most preferably, the effective daily dosage is administered between once daily and four times daily in dosage units of accordingly adjusted weight. While not limiting to the inventive subject matter, acetyl-L-carnitine in contemplated formulations is thought to increase cerebral energy metabolism by assisting in mitochondrial beta-oxidation and to donate an acetyl moiety for synthesis of acetylcholine, while Vinpocetine is thought to dilate blood vessels in the brain, as well as improve red blood cell deformability, to thus allow for better perfusion into and throughout neuro-cognitive regions and structures of the brain. Huperzine A is thought to act as an acetylcholine esterase inhibitor and antioxidant. Alpha lipoic acid is thought to act as an anti-oxidant with significant action at the mitochondrial level, and in the regeneration of glutathione and other anti-oxidants, such as, vitamin E, and also to act as a facilitator of glucose utilization, while Rhodiola is considered to increase the catecholamine-type transmitters and to reduce adverse effects of corticosteroids, and particularly cortisol. Biotin is thought to improve glucose utilization and tolerance, thus supporting brain metabolism that predominantly employs glucose as an energy source, and to replace natural biotin stores in the body that can be reduced by the intake of alpha lipoic acid.

In still further contemplated aspects, the nutritional supplement may further be associated with an information that states that the nutritional supplement enhances at least one of working memory, general intelligence, attention, and mood. For example, such information may be provided as a packaging insert, a label on a container that comprises the supplement, and/or on a sales display. Alternatively, or additionally, information may also be provided as displayed information (e.g., on website, or TV commercial) or as audible information. Moreover, in further contemplated aspects, the supplement may be provided with an interactive tool that allows validation of efficacy of the supplement and/or proper personal dosing of the supplement. Such interactive tool may be software on a data carrier, printed information (e.g., a booklet), or a link to a website hosting the tool.

Remarkably, the composition according to the inventive subject matter had a markedly shifted activity profile for cognitive enhancement due to adding selected additional ingredients to a clinically effective base formulation (also referred to herein as “Procera”). Clinical studies of Procera (Huperzine A, Vinpocetine and Acetyl-L-Carnitine at amounts as shown below) indicated improvements in both cognition and mood in healthy participants aged 25-60 years. Statistically significant improvements in several variables relative to placebo could be attributed to the 4 week administration of Procera. Specifically, Numerical Working Memory Accuracy (working memory), Word Recognition Speed (long term memory consolidation), Anger-Hostility (mood), Total Mood Disturbance (mood), and Raven Progressive Matrices (fluid intelligence, spatial and object working memory) improved. The study also found some evidence approaching statistical significance of the following measures to be improved due to the 4 week Procera treatment: Spatial Working Memory Accuracy (working memory), Depression (mood), Confusion (mood), and Vigor (mood).

A preferred composition according to the inventive subject matter (also referred to herein as “Ceretrophin”, which includes Huperzine A, Vinpocetine Acetyl-L-Carnitine, Rhodiola, R-Alpha Lipoic Acid and Biotin at amounts as indicated below) also improved both cognition and mood in a clinical study of healthy participants aged 25-60 years, however, with a substantially distinct activity profile. Statistically significant improvements could be attributed to the 4 week administration of Ceretrophin for Raven Progressive Matrices (working memory, general intelligence), Digit Vigilance Errors (attention), and Stress (mood). The study also found some evidence approaching statistical significance of the following measures to be improved due to the 4 week Ceretrophin treatment: Spatial Working Memory Errors (working memory), Numerical Working Memory Accuracy (working memory), Word Recognition Accuracy Original Stimuli (memory consolidation), and Tension (mood).

### EXPERIMENTS

### Clinical Study of Inventive Composition (Ceretrophin) vs. Comparative Composition (Procera) and Result Summary

The study was conducted to provide evidence on whether one month administration of the inventive composition improves cognitive functioning in healthy human participants. Approximately 100 participants were initially enrolled into the clinical study. Human cognition is complex, but can be measured using standardized tests of information processing, reaction time, attention, concentration, working memory, long term memory and decision making. These standardized measures relate to one performs simple and complex tasks in real life. By assessing a range of cognitive measures before and after one month administration of either the inventive composition or placebo it is possible to gauge whether the inventive composition has a positive effect in improving human cognition.

The study was a randomized, double-blind, placebo controlled study examining the effects of Ceretrophin vs placebo on cognitive function and mood (a substantially identical study was performed examining the effects of Procera vs placebo to allow for a comparison between Ceretrophin and Procera). This means that the participants were randomly allocated to either a placebo or Ceretrophin group in which they were administered either placebo or Ceretrophin tablets for one month. The study was double blind because both the experimenters and the participants did not know which tablets they were taking.

In the following, the composition according to the inventive subject matter is referred to as Ceretrophin, while the comparative composition was termed Procera. Remarkably, while both compositions produced significant cognitive effects in various arenas, the actual effects differed in specific areas in a statistically significant manner.

Ceretrophin Composition (daily dose in mg)

| | |
|---|---|
| Huperzine A | 0.15 |
| Vinpocetine | 15 |
| Acetyl-L-Carnitine | 1500 |
| Alpha Lipoic Acid | 400 |
| Rhodiola rosea (standardized to 3% Rosavin) | 300 |
| Biotin | 0.50 |

Comparative Composition (Procera; daily dose in mg)

| | |
|---|---|
| Huperzine A | 0.15 |
| Vinpocetine | 15 |
| Acetyl-L-Carnitine | 1500 |

As can be seen, the inventive composition employs additional ingredients over the three other ingredients (Huperzine A, Vinpocetine, and Acetyl-L-carnitine). Otherwise, formulation in tablets for oral administration of about 500 mg was identical, however, the improvements in cognitive and mood measures was markedly different.

Specifically, while Procera exhibited a greater number of memory markers that showed significant improvements, Ceretrophin provided a significant (P=0.001) and substantial gain in “IQ” (about 6 points, or about 0.3 SD) as measured by the Ravens Progressive Matrices, which is a conventional IQ (Wechsler) test that loads more on fluid intelligence, reasoning and spatial working memory. Ceretrophin also showed stronger improvements in attention, or concentration. While not wishing to be bound by any theory or hypothesis, the inventor contemplates that Ceretrophin adds additional neurotransmitter (catecholaminergic) support for alertness via the addition of Rhodiola extract. This might also explain its apparently significant impact on mood (and particularly stress). Of further note is the fact that Ceretrophin seemed to perform better at higher cognitive loads or task complexity. However, why Ceretrophin did not perform as well as Procera in various measures of memory is not clear. Thus, it should be especially appreciated that seemingly subtle changes in composition of a nootropic formulation may result in various statistically significant, remarkable, and unexpected differences, which was further confirmed by the complete ineffectiveness of yet further seemingly similar compositions (see below).

It should also be especially appreciated that the study was conducted with cognitively normal male and female subjects ranging in age form 25-60. Producing an overall effect in such a large demographic group is unexpected. Rather, if anything (and based on results for individual components), it could be expected that the type of formulation used herein would only show significant improvements in either a slightly to moderately impaired group and an older group (e.g., age 45 plus), where many conditions of aging and lifestyle factors contribute to an accumulated buildup of neurotoxic factors (e.g., free radical induced oxidative stress, heavy metals, cerebral vascular plaques, including beta amyloid plaques implicated in Alzheimer's, reduced cerebral vascular blood flow and glucose metabolism, calcium dyshomeostasis and others).

### Methods

Participant Selection Criteria; Selection criteria includes those: (1) Not currently taking prescription drugs affecting the brain or nervous system (e.g., Modafinil, acetylcholinesterase inhibitors, anti-cholinergics, stimulants, L-dopa, MAO inhibitors, NMDA receptor antagonists, methylphenidate, amphetamine, pseudo-ephedrine, SSRIs and other anti-depressant medication); (2) Not currently taking OTC medications affecting the brain (e.g., ephedra based diet pills); (3) Who have not used any supplements within the past 30 days that have an effect on cognitive function, memory, anxiety, depression (e.g. Ginseng, Gingko, Vinpocetine, 5HTP, Tryptophan, St. John's Wort, ephedrine (ephedra), alpha GPC, Citicoline, phosphatidylserine, acetyl-l-carnitine, Focus Factor™); (4) Not active Smokers; (5) Not taking the following: anti-coagulant drugs (Warfarin, Heparin, Plavix); anticholinergics or acetylcholinesterase inhibitors (bethanechol (Ureholine), donepezil (Aricept), rivastigmine (Exelon), galantamine (Reminyl), edrophonium (Enoln, Reversol, Tensilon), neostigmine (Prostigmin); (6) Do not have any of the following health conditions: AIDS, HIV; Chronic Fatigue Syndrome, Epstein Barr, Fibromylagia, Lupis, Multiple Sclerosis, Thyroiditis, Ulcerative Colitis, Crohn's Disease, Irritable Bowel Syndrome, dementia including Alzheimer's and Parkinsons' disease, Type 1 or 2 Diabetes, Insomnia or Sleep Apnea, Narcolepsy; (7) No history of head trauma; (8) No neurological deficits; (9) Not pregnant or lactating; (10) Not anticipating any planned changes in lifestyle (e.g. exercise regimen) for the duration of the study; (11) No known allergies to nuts.

50 healthy participants between the ages of 25-65 years of age, were tested in treatment and placebo groups (total number of participants is n=100). A drop out rate (voluntary and non-voluntary withdrawal) of approximately 20% was expected and therefore additional participants were recruited for the study. All interested individuals were screened over the phone by a research nurse to assess their suitability for participation in the study. Subjects participated in periodic evaluation of their cognitive functions including memory, mood, energy and mental status by taking a battery of computerized tests and written questionnaires that assessed their cognitive functions which including attention, memory, executive function, mood, energy, stress level, state of mind and IQ.

The following neuropsychological tests were employed in the currents study: (1) The Cognitive Drug Research measure (CDR) is a well-validated test, which was used to assess attention, working memory and episodic secondary (longer term memory, or consolidation). (2) Inspection time (IT) is a measure speed of early information processing. (3) The Profile of Mood States (POMS) is a self-report designed to measure six dimensions of mood: tension-anxiety; depression-dejection; anger-hostility; vigor-activity; fatigue-inertia; and confusion-bewilderment. (4) IQ was assessed using the Raven's Progressive Matrices. This was done by administering the even items at baseline and the odd items at Week 4. (5) The UWIST Mood Adjective Checklist was used to measure mood states and energy levels. (6) The Spielberger State-Trait Anxiety Inventory is a 20-item questionnaire, to measure anxiety at the time of testing. (7) Perceived Stress Scale was used to measure stress symptoms and effective coping.

Participants visited Swinburne University, Melbourne, Australia, on 3 separate occasions Visit 1: Health assessment, practice, baseline and acute testing; Visit 2:1 week (7 days) following baseline testing; and Visit 3:4 weeks (28 days) following baseline testing. During the first visit, participants completed a general health assessment and were then allocated into one of three treatment groups for baseline and acute testing.

### (1) Cognitive Measures

Raven Progressive Matrices (general intelligence IQ): Participants in the Ceretrophin group statistically improved their performance on the Raven Progressive Matrices relative to the placebo group (p<0.001). This was a very strong effect and equates to an IQ improvement of about 6 IQ points. The Raven Progressive Matrices is a well-validated non-verbal measure of general intelligence. To complete this task a participant must engage in several higher-order cognitive processes such as visualization, spatial working memory, mental rotation, reasoning, and non-verbal problem solving. This is a remarkable result, particularly given the statistical significance and effect size. This result supports the smaller improvements in accuracy of the less difficult tasks used in the CDR battery. It is of note that the most significant effect of Ceretrophin is seen with the most complex task.

| | | | Std. | |
|---|---|---|---|---|
| | Condition | Mean | Deviation | N |
| Raven's - | Ceretrophin | 8.2500 | 3.34984 | 36 |
| advance | Placebo | 9.3929 | 3.77457 | 28 |
| progressive | Total | 8.7500 | 3.55903 | 64 |
| matrix - | | | | |
| baseline | | | | |
| Raven's - | Ceretrophin | 9.7500 | 3.47542 | 36 |
| advance | Placebo | 8.1786 | 4.49735 | 28 |
| progressive | Total | 9.0625 | 3.99950 | 64 |
| matrix - week 4 | | | | |

Simple Reaction Time: The speed of simple reaction time did not significantly improve due to the Ceretrophin treatment across the 4 weeks of administration. This is the simplest cognitive measure in the cognitive battery. This result is consistent with the results from the other main variables in so far as the Ceretrophin did not speed up neural processes but instead improved accuracy and reduced mistakes.

Digit Vigilance and Choice Reaction Time: The Ceretrophin treatment significantly (p=0.05) decreased the number of false alarms (mistakes) during the Digit Vigilance task after 4 week administration. Participants in the Ceretrophin group relative to the placebo group improved their attention/concentration. This was a relatively strong effect.

| | | | Std. | |
|---|---|---|---|---|
| | Condition | Mean | Deviation | N |
| Digit Vigilance - | Ceretrophin | 1.0513 | 1.19095 | 39 |
| False Alarms - | Placebo | .6129 | 1.05443 | 31 |
| BASELINE | Total | .8571 | 1.14570 | 70 |
| Digit Vigilance - | Ceretrophin | .7436 | .78532 | 39 |
| False Alarms - | Placebo | .7742 | 1.02338 | 31 |
| Week 4 | Total | .7571 | .89176 | 70 |

Performance on the Choice Reaction Time Accuracy also improved due to the Ceretrophin and this result approached statistical significance (p=. 1). The effects of the Ceretrophin was not to speed up the brain directly or to make participants quicker to respond to the discrimination, but gave them better accuracy in discriminating between the stimulus alternatives. This indicates an improvement in the efficiency of decision making and information processing. Note that there was not a slowing of reaction time (RT), which led to an increase in accuracy. The increase in accuracy due to the Ceretrophin was not a consequence of a slowing of response time (increase in RT). Although approaching statistical significance this was not a strong effect.

| | | | Std. | |
|---|---|---|---|---|
| | Condition | Mean | Deviation | N |
| Choice Reaction | Ceretrophin | 96.8421 | 2.73640 | 38 |
| Time - Accuracy - | Placebo | 97.4000 | 2.58110 | 30 |
| baseline | Total | 97.0882 | 2.66394 | 68 |
| Choice Reaction | Ceretrophin | 97.3158 | 2.42849 | 38 |
| Time - Accuracy - | Placebo | 97.0667 | 3.51287 | 30 |
| Week 4 | Total | 97.2059 | 2.93491 | 68 |

Spatial Working Memory: There was a trend towards significance for Spatial Working Memory Outliers (p=0.13). Although not significant, the results (see mean values below) indicate that there was more of an improvement in the number of mistakes over the treatment duration for the Ceretrophin than for the placebo. Larger sample size may help this result become statistically significant. This result should be treated as a preliminary finding that should be validated in a larger sample.

| | | | Std. | |
|---|---|---|---|---|
| | Condition | Mean | Deviation | N |
| Spatial Working | Ceretrophin | 1.0000 | 1.16190 | 41 |
| Memory - Outliers - | Placebo | .7813 | .83219 | 32 |
| baseline | Total | .9041 | 1.02962 | 73 |
| Spatial Working | Ceretrophin | .7317 | 1.04939 | 41 |
| Memory - Outliers - | Placebo | .9063 | 1.20106 | 32 |
| week 4 | Total | .8082 | 1.11377 | 73 |

Numerical Working Memory: Participants on the Ceretrophin treatment showed an improvement (p=0.18) in Numerical Working Memory Accuracy compared to placebo participants. This again approached statistical significance. The result indicates that there is some evidence that there is an improvement in holding numbers in working memory (immediate memory) from Baseline to Week four due to the Ceretrophin treatment. Increasing the sample size (statistical power) may result in this variable showing statistical significance. This is an interesting but preliminary finding.

| | | | Std. | |
|---|---|---|---|---|
| | Condition | Mean | Deviation | N |
| Numeric Working | Ceretrophin | 92.1645 | 7.24746 | 38 |
| Memory Original | Placebo | 95.7787 | 5.26386 | 30 |
| Stimuli - Accuracy - | Total | 93.7590 | 6.65344 | 68 |
| baseline | | | | |
| Numeric Working | Ceretrophin | 92.6326 | 7.80322 | 38 |
| Memory Original | Placebo | 95.0380 | 3.94556 | 30 |
| Stimuli - Accuracy - | Total | 93.6938 | 6.46620 | 68 |
| week 4 | | | | |

Picture Recognition There was no significant change in performance in Picture Recognition over the 4 week trial attributable to either Placebo or Ceretrophin treatment.

Word Recognition: Word Recognition Accuracy improved for the Ceretrophin participant group but decreased for the Placebo participant group across the 4 weeks of the trial. Although this only approached statistical significance (p=0.12) the results provides some evidence that Ceretrophin treatment improves the accuracy of memory consolidation of words. Again, a systematic picture of results is emerging with many variables showing improvement in accuracy rather than speed, and that this improvement in accuracy is not a consequence of a slowing of RT (or more cautious responding). Overall the changes to the different accuracy variables suggest that the Ceretrophin improves efficiency by reducing the number of errors of neural processing of cognitive measures.

| | | | Std. | |
|---|---|---|---|---|
| | Condition | Mean | Deviation | N |
| Word | Ceretrophin | 73.3336 | 16.25226 | 36 |
| Recognition | Placebo | 74.2534 | 14.87757 | 29 |
| Original Stimuli - | Total | 73.7440 | 15.54023 | 65 |
| Accuracy - | | | | |
| baseline | | | | |
| Word | Ceretrophin ™ | 75.1853 | 15.50148 | 36 |
| Recognition | Placebo | 73.1038 | 14.19566 | 29 |
| Original Stimuli - | Total | 74.2566 | 14.85472 | 65 |
| Accuracy - | | | | |
| week 4 | | | | |

Inspection Time: A smaller sub-set of participants completed this task. No differences were observed between the Ceretrophin and placebo groups but this may be due to the low sample size.

### (2) Mood Measures

Perceived Stress (p<0.05): Four week treatment of Ceretrophin showed a small reduction in the levels of stress perceived by participants relative to the placebo group. It is noteworthy that participant recruitment did not involve highly stressed or anxious individuals but just normal population levels of stress and other moods.

| | | | Std. | |
|---|---|---|---|---|
| | Treatment | Mean | Deviation | N |
| Perceived | Ceretrophin | 28.2973 | 3.02641 | 37 |
| Stress Scale | Placebo | 29.0333 | 3.87283 | 30 |
| baseline | Total | 28.6269 | 3.42378 | 67 |
| Perceived | Ceretrophin | 27.4054 | 3.24431 | 37 |
| Stress Scale | Placebo | 29.6333 | 3.83705 | 30 |
| Week 4 | Total | 28.4030 | 3.66829 | 67 |

Tense Arousal (p=0.12): Consistent with the reduction in the level of stress, we observed a reduction in the level of tense arousal. However, this result was not statistically significant and a larger sample would increase the statistical power with this variable.

| | | | Std. | |
|---|---|---|---|---|
| | Treatment | Mean | Deviation | N |
| tense arousal | Ceretrophin | 26.8421 | 3.90790 | 38 |
| baseline | Placebo | 26.8065 | 4.30828 | 31 |
| (UMACL) | Total | 26.8261 | 4.06186 | 69 |
| tense arousal | Ceretrophin | 25.4737 | 5.43132 | 38 |
| Week 4 | Placebo | 26.8710 | 4.90403 | 31 |
| (UMACL) | Total | 26.1014 | 5.21069 | 69 |

In terms of cognitive variables, there is evidence that Ceretrophin improves functioning during highly complex cognitive tasks that assess general reasoning and problem solving. There was also some evidence that Ceretrophin improved working memory variables. The results if taken together do also suggest an improvement in the efficiency of information processing and decision making such as in improving accuracy and reducing cognitive errors. The reduction in errors and improvement in accuracy was seen in nearly all tasks. The highly statistically significant improvement in general intelligence from the Raven Progressive Matrices was larger than the other cognitive variables and so was easily statistically observed.

In terms of mood, Ceretrophin appears to reduce stress and tension. Given the increase in occupational stress seen throughout the western world, this is an important finding. A larger sample would be useful in better understanding the changes in mood due to treatment in Ceretrophin.

Overall the results suggest that Ceretrophin is a unique composition that exerts beneficial effects to both cognition and mood, particularly in general intelligence and during complex cognitive reasoning tasks/decision making. Statistically significant improvements in several variables relative to placebo could be attributed to the 4 week administration of Ceretrophin in the Raven Progressive Matrices (working memory, general intelligence), Digit Vigilance Errors (attention), Stress (mood). The study also found some evidence (approaching statistical significance) of the following measures to be improved due to the 4 week Ceretrophin treatment: Spatial Working Memory Errors (working memory), Numerical Working Memory Accuracy (working memory), Word Recognition Accuracy Original Stimuli (memory consolidation), and Tension (mood).

As indicated above, substantially similar investigative and analytic methods were used for Procera, and the exact protocol is described in my copending U.S. patent application with the title “Compositions And Methods For Enhancing Brain Function” (inventor J. Reynolds), filed on Jun. 5, 2007, which is incorporated by reference in its entirety.

### Additional Comparative Examples

The following additional examples are provided to illustrate the unexpected significant differences in modulation of various parameters of cognition in light of relatively moderate changes in composition of an orally administered supplement with suspected nootropic effect. Formulation C had vinpocetine and various B-vitamins, while Formulation E had vinpocetine, huperzine A, acetyl-L-carnitine, in amounts relatively similar to those of Procera and Ceretrophin, and four other active ingredients (Vitamins Nicain, Thiamine, and Pantothenic Acid, and DMAE). Remarkably, none of the below provided additional comparative examples resulted in apparent and statistically significant improvement of any of the tested cognitive parameter.

Formulation C

| | |
|---|---|
| Vinpocetine | 20 mg |
| Pantothenic Acid (Vit. B-5) | 250 mg |
| Dimethylaminoethanol | 300 mg |
| Thiamin (Vit. B-1) | 250 mg |
| Niacin (Vit. B-3) | 20 mg |

Formulation E

| | | |
|---|---|---|
| Huperzine A | 150 | mcg |
| Vinpocetine | 20 | mg |
| Pantothenic Acid (Vit. B-5) | 250 | mg |
| Dimethylaminoethanol | 300 | mg |
| Thiamin (Vit. B-1) | 250 | mg |
| Niacin (Vit. B-3) | 20 | mg |
| Acetyl L Carnitine | 1000 | mg |

Chronometric (brain speed) testing can identify what information processing stage is impacted by the therapeutic agent. This may include: motor reflexes (physical reaction time); perceptual acuity; executive function (decision-making speed) and attention; alertness; mental agility (fluid intelligence), and memory (immediate & delayed).

The following CogCAM™ tests were used: CogCAM 4 Working Memory Speed (decision-making; task-shifting); CogCAM10A Memory Scanning (semantic; letters); CogCAM 10B Memory Scanning (visual-spatial; symbols); CogCAM 1 physical reflexes (simple reaction time; attention). These tests provide primary measures of attention, memory and executive cognitive function.

Inclusion Criteria: Male, and non-pregnant (self reported) female subjects, 18 years of age or older, no planned change in lifestyle including exercise regimen during study.

Exclusion Criteria: 1. Taking prescription drugs affecting the brain or nervous system within two weeks of study entry (e.g., epilepsy, Alzheimer's disease, Parkinson's disease, anxiety, depression, psychosis, ADD or other psychiatric condition); 2. Taking OTC medications affecting the brain within two weeks of study entry (e.g. diet pills); 3. Taking supplements known to have an effect on cognitive function, memory, anxiety, depression within two weeks of study entry (e.g., Arctic root or Rhodiloa, Ginseng, Gingko, Vinpocetine, 5HTP, St. John's wort, ephedrine (ephedra), phosphatidyl choline, phosphatidyl serine, alpha GPC, acetyl-l-camitine); 4. Smokers

### Methods and Results

To assess the influence of multiple formulations of Formulation C and E on cognitive function, a battery of web-based tests (the Cognometer) was administered over a 6-week treatment period. An analysis of the data compared baseline performance to subsequent weekly exams with placebo or 1 of 5 formulations taken daily to determine if there was a change in cognitive function after initiation of treatment with the dietary supplement compounds in cognitively intact individuals 18 to 74 years of age. Placebo (Group A) was a sugar pill.

Cognitive performance measures were obtained from web-based assessments using the Cognometer test battery subtests; 4-Executive function and 10-Immediate Memory. There were 2 treatment groups and one control group in this study. The group conditions remained blinded in these analyses, the analyses were completed without knowledge of which groups received the test compounds or placebo. Only individuals who completed tests in each of week (e.g., baseline and all dosing weeks) were included in the analysis. Outliers who scored more than two standard deviations from the mean on a test, and were not internally consistent with other test scores were also eliminated. The elimination of outliers was done to avoid including results that may be due to distractions or web/computer glitches that could invalidate the particular test session. Analysis of the data uses an analysis of variance (ANOVA) for the differences between the baseline and last week of treatment.

### Results And Statistical Analysis

The trial used the Internet to recruit, qualify, register, and test over 1000 subjects with 430 completing the 6 week study. Testing was conducted at week 0, Baseline, and every subsequent testing and reporting week for 6 weeks during which subjects were administered the test formulations. On each test day subjects also completed adverse event forms, questionnaires concerning any changes in lifestyle factors, and cognitive testing. Following is a description of the Cognometer Tests 4 and 10 used in the testing of the compounds and the interpretative data and possible claims that improvement in these tests represent and support.

Test 4 is a “complex choice reaction time task” that tests so called executive cognitive function, or decision making performance speed measured in milliseconds (ms). It has an added unique feature of a random rule reversing cue which tests both one's ability to rapidly inhibit one mode of response and switch to another response mode, considered a higher order cognitive function. Facility in “inhibition and task shifting” can be equated to mental flexibility. Improvement in reaction time on this test supports the claims of: improved mental quickness and flexibility; improved decision making; improved decision making speed; improved cognitive processing; improved decision making speed in a demanding cognitive task.

Test 4 RT data can also be analyzed to assess the group's level of focus, or attention. This measure is derived from computing the standard deviation of the individual's intra trial reaction times (RTSD). This basically represents the consistency of their responses (processing efficiency) and is considered to reflect the level of sustained attention. Improved performance on this score, that is RTSD, supports claims of: improved attention or focus; improved attention or focus on a demanding cognitive task.

Test 10 is divided into two tests, recognition recall of letters and spatial patterns. Only the visuo-spatial memory part of this test showed significance. This test is patterned after the Sternberg Memory Scan paradigm wherein immediate and short term memory processing (scanning & recall) speed equates to memory encoding. Sternberg-like tests, like Cognometer Test 10, have been used for over 30 years in clinical trials and pharmaceutical research to determine drug effects on memory processes. Improved reaction times on this test support claims of: improved memory; improved memory processing speed; improved encoding of information; improved recall speed.

The most notable, however, statistically insignificant effects were found in the 35 plus age group, probably suggesting that the compounds may be effective in those who are beginning to exhibit normal age related slowing associated with increased years of life, typically after 30 years of age. Reaction time standard deviation (RTSD) for this test of executive function did not show a significant difference between groups. The reaction time median scores (RTmed), a measure of executive function (decision making and mental flexibility) did also not indicate significant between group differences.

Selected results are as follows, with Group A taking placebo, Group C taking Formulation C, and Group E taking C Formulation E.

RT Median - Test 4

| Group | Mean | SEM |
|---|---|---|
| A | 91.509 | 2.498 |
| C | 88.315 | 2.343 |
| E | 94.505 | 2.352 |

RT Standard deviation - Test 4

| Group | Mean | SEM |
|---|---|---|
| A | 85.045 | 5.695 |
| C | 89.4 | 4.588 |
| E | 88.8 | 5.506 |

RT Median (shapes) - Test 10

| Group | Mean | sem |
|---|---|---|
| A | 91.523 | 2.016 |
| C | 95.698 | 2.797 |
| E | 93.103 | 2.093 |

Thus, no significant improvement in cognitive functions was observed with those formulations tested. Such finding is once more remarkable as the formulations appear to have similar compositions to the Ceretrophin composition, but significantly different effects in toto.

Thus, specific embodiments of modified nutritional supplements for enhancing cognitive function have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A nutritional supplement for enhancing cognitive function, comprising a group of active ingredients consisting of huperzine A, vinpocetine, acetyl-L-carnitine, alpha lipoic acid, rhodiola, and biotin.

2. The nutritional supplement of claim 1 wherein the huperzine A, the vinpocetine, and the acetyl-L-carnitine are present in a ratio of x:y:z, wherein x is between 0.8 and 1.2, y is between 80 and 120, and z is between 8,000 and 12,000.

3. The nutritional supplement of claim 1 wherein a ratio between the acetyl-L-carnitine and the alpha lipoic acid, the rhodiola, and the biotin is between 2:1 and 20:1, between 2:1 and 20:1, and between 20,000:1 and 200:1, respectively.

4. The nutritional supplement of claim 1 wherein in a daily dose huperzine A is present in an amount between 100-200 mcg, vinpocetine is present in an amount between 10-30 mg, and acetyl-L-camitine is an amount between 1,500-2,000 mg.

5. The nutritional supplement of claim 1 wherein in a daily dose alpha lipoic acid is present in an amount between 250-500 mg, rhodiola is present in an amount between 200-400 mg, and biotin is present in an amount between 250-750 mcg.

6. The nutritional supplement of claim 4 wherein in a daily dose alpha lipoic acid is present in an amount between 250-500 mg, rhodiola is present in an amount between 200-400 mg, and biotin is present in an amount between 250-750 mcg.

7. The nutritional supplement of claim 1 further comprising an inactive ingredient is selected from the group consisting of a carrier, a binder, an excipient, and a dye.

8. The nutritional supplement of claim 1 wherein the huperzine A, vinpocetine, and acetyl-L-camitine together account for at least 50 wt % of a dosage unit of the supplement, excluding inactive ingredients.

9. The nutritional supplement of claim 1 wherein the supplement is formulated for oral administration such that the group of active ingredients accounts for at least 85 wt % of a dosage unit of the supplement.

10. The nutritional supplement of claim 1 wherein the supplement is formulated for oral administration such that the group of active ingredients accounts for at least 90 wt % of a dosage unit of the supplement.

11. The nutritional supplement of claim 1 wherein the daily dosage of the supplement is equal or less than 2,300 mg.

12. The nutritional supplement of claim 1 further comprising an information stating that the nutritional supplement enhances at least one of working memory, general intelligence, attention, and mood.

13. A method of assisting enhancement of cognitive function in a person using a nutritional supplement, comprising:
providing a composition that includes a group of active ingredients consisting of huperzine A, vinpocetine, acetyl-L-carnitine, alpha lipoic acid, rhodiola, and biotin;
formulating the supplement for oral administration such that the active ingredients together account for at least 90 wt % of a dosage unit of the supplement, excluding inactive ingredients; and
providing an information that the supplement in a clinical trial was effective to improve cognitive function.

14. The method of claim 13 wherein the huperzine A, the vinpocetine, and the acetyl-L-carnitine are present in a ratio of x:y:z, wherein x is between 0.8 and 1.2, y is between 80 and 120, and z is between 8,000 and 12,000.

15. The method of claim 13 wherein a ratio between the acetyl-L-carnitine and the alpha lipoic acid, the rhodiola, and the biotin is between 2:1 and 20:1, between 2:1 and 20:1, and between 20,000:1 and 200:1, respectively.

16. The method of claim 13 wherein in a daily dose huperzine A is present in an amount between 100-200 mcg, vinpocetine is present in an amount between 10-30 mg, and acetyl-L-carnitine is an amount between 1,500-2,000 mg.

17. The method of claim 13 wherein in a daily dose alpha lipoic acid is present in an amount between 250-500 mg, rhodiola is present in an amount between 200-400 mg, and biotin is present in an amount between 250-750 mcg.

18. The method of claim 16 wherein in a daily dose alpha lipoic acid is present in an amount between 250-500 mg, rhodiola is present in an amount between 200-400 mg, and biotin is present in an amount between 250-750 mcg.

19. The method of claim 13 wherein the cognitive function is at least one of working memory, general intelligence, attention, and mood.

20. The method of claim 13 further comprising a step of providing an interactive tool that allows at least one of validation of efficacy of the supplement and proper personal dosing of the supplement.
